(19) United States
(12) Patent Application Publication
(10) Pub. No.: US 2026/0048229 A1
(43) Pub. Date: Feb. 19, 2026

(54) Urinary Catheter

(71) Applicant: The Flume Catheter Company Limited, Farnham (GB)

(72) Inventors: Kristian Olsen, Salt Lake City, UT (US); Roger Anthony Holmes, Surrey (GB); John Spencer Havard, Suffolk (GB)

(21) Appl. No.: 19/230,426

(22) Filed: Jun. 6, 2025

Related U.S. Application Data
(63) Continuation-in-part of application No. PCT/GB2025/050942, filed on May 2, 2025.
(60) Provisional application No. 63/641,507, filed on May 2, 2024.

(51) Int. Cl. A61M 25/00; A61L 29/06; A61M 25/10

(52) U.S. Cl. CPC A61M 25/0017; A61L 29/06; A61M 25/0009; A61M 25/1025; A61M 25/1027; A61M 2205/0216

(57) ABSTRACT

A urinary catheter has a shaft having a proximal end and a distal end, the shaft having a lumen extending between the proximal end and the distal end; and a port unit secured to the distal end of the shaft, the port unit comprising a port configured to communicate with the lumen of the shaft via a passage in the port unit, the distal end of the shaft being received in a recess of the port unit.

### CROSS-REFERENCE TO RELATED APPLICATIONS AND CLAIM OF PRIORITY

This application is a continuation-in-part under 35 U.S. C. 120 and 35 U.S. C. 363 of copending Application No. PCT/GB2025/050942 filed May 2, 2025, which designated the US, which claims priority under 35 U.S.C. 365 from US Provisional Application Ser. No. 63/641,507 filed May 2, 2024, the contents of which are incorporated by reference herein in their entireties.

### FIELD OF THE INVENTION

This invention relates to urinary catheters.

### BACKGROUND

Urinary catheters are used to assist or control the flow of urine from the bladder of a patient. When a patient needs to use a catheter for an extended period of time, they may use an indwelling urinary catheter. An indwelling urinary catheter has a tube which is introduced through the patient's urethra or directly via an abdominal incision (supra-pubic catheter). Once the tip of the catheter is in the bladder it is retained in position by means such as a balloon inflated within the bladder. A lumen extending through the catheter can then drain urine from the bladder.

A common design of indwelling urinary catheter is the Foley catheter. In the Foley catheter, the balloon is toroidal in shape and is located proximally of the catheter tip. A drainage port which communicates with the lumen is located between the catheter tip and the balloon. Catheters of this design suffer from a number of problems. The tip of the catheter is exposed and can irritate the bladder wall. Material of the bladder wall can become drawn into the drainage port, causing discomfort and mucosal damage. The drainage port is spaced from the base of the bladder by the balloon, which prevents the bladder draining completely leading to a residual pool of urine that can become infected.

WO 2018/134557 discloses one approach for addressing at least some of these problems. It provides a urinary catheter having an inflatable balloon in the form of an elongate tube extending over the tip and providing the drainage port on a side of the shaft.

However, this approach is open to further improvement to allow for alternative routes of manufacture for urinary catheters.

### SUMMARY OF THE INVENTION

According to one aspect, there is provided a unitary catheter comprising: a shaft having a proximal end and a distal end, the shaft having a lumen extending between the proximal end and the distal end; and a port unit secured to the distal end of the shaft, the port unit comprising a port configured to communicate with the lumen of the shaft via a passage in the port unit, the distal end of the shaft being received in a recess of the port unit.

The catheter may further comprise a balloon installed on the port unit.

The balloon may be in the form of an elongate tube.

At least part of the balloon may be located proximally of the port.

The distal end of the shaft may terminate at a plane perpendicular to the longitudinal axis of the shaft.

The port may be a drainage port. The passage may be a drainage passage. The lumen may be a drainage lumen of the shaft.

The port may be on a side of the port unit.

The port unit may comprise an inflation port configured to communicate with an inflation lumen of the shaft via an inflation passage in the port unit.

The port unit may comprise one or more of a first shoulder and a second shoulder. The radius of the port unit may reduce at each shoulder. The inflation port may be on the first shoulder.

There may be a connection tube in the inflation passage for providing an inflation path between the inflation lumen and the balloon. The connection tube may be made from a polymer, such as polycarbonate.

The connection tube may protrude from the inflation port.

The urinary catheter may further comprise an adhesive for securing the shaft to the port unit. The adhesive may be a silicone adhesive. The silicone adhesive may be moisture cured.

There may be a bonding layer between the shaft and the port unit. The bonding layer may comprise an adhesive. The bonding layer may comprise a zone of melted or softened material by which the shaft and the port unit have been heat welded together.

The adhesive may secure the connection tube to the inside of the inflation lumen of the shaft and to the inflation passage of the port unit.

The adhesive may secure the connection tube to the interior of the balloon.

The urinary catheter may further comprise a heat shrink component for smoothing a transition region between the shaft and the port unit.

The port unit may be formed of a material comprising a silicone elastomer.

The port unit may be an injection moulded component.

The recess of the port unit may comprise a proximal flange hugging the distal end of the shaft.

The recess of the port unit may hug or surround the distal end of the shaft around an arc of more than 180 degrees about the longitudinal axis of the shaft.

The distal end of the shaft may extend into the recess of the port unit by at least 2 mm.

The balloon may comprise a first region secured to the port unit, a second region secured to the port unit and an elastic-walled and/or flexible-walled conduit extending between the first region and the second region. The balloon may extend over the tip of the port unit. The elastic-walled conduit may extend over the tip of the port unit. The balloon may comprise two walls where it extends over the tip of the port unit. The balloon may be configured such that when inflated an exterior wall of the balloon is located distally of and spaced from the tip. The balloon may be configured such that when inflated an interior wall of the balloon bears against the tip.

The shaft may be an elongate shaft.

The catheter may be formed of two components. The shaft may be a first component. The port unit may be a second component.

The shaft and the port unit may be formed separately. The shaft and the port unit may be formed of different materials. In other words, the shaft may be formed from a different material to the material of the port unit.

The balloon may be in the form of an elongate tube and may be secured to the port unit between first and second ends of the elongate tube. The balloon may be secured to the port unit along at least 50%, 60%, 70%, 80% or 90% of the length of an internal wall of the balloon. The balloon may be secured to the port unit along the entire length of the internal wall. The balloon may be secured to the port unit using an adhesive, for example a silicone adhesive.

According to another aspect, there is provided a port unit for a urinary catheter comprising: a recess configured to receive a catheter shaft; and a port configured to communicate with a lumen of the catheter shaft via a passage in the port unit.

The port unit may have any of the features disclosed herein.

According to a further aspect, there is provided a method for manufacturing a urinary catheter comprising: providing a shaft having a proximal end and a distal end; providing a port unit, the port unit having a recess for receiving the distal end of the shaft and a port for communicating with a lumen of the shaft via a passage in the port unit; and securing the port unit to the distal end of the shaft with the distal end of the shaft received in the recess.

The method may further comprise: providing a balloon in the form of an elongate tube; providing a connection tube; sealing the elongate tube at a first end; sealing the elongate tube around the connection tube at a second end; advancing the connection tube through an inflation passage of the port unit to connect the connection tube with an inflation lumen of the shaft; and bonding the connection tube to the inflation lumen. The method may further comprise shrinking a heat shrink component over a transition between the shaft and the port unit so as to smooth the transition.

The method may further comprise, prior to securing the port unit to the distal end of the shaft, forming the shaft by extrusion and/or forming the port unit by injection moulding.

In some embodiments, the catheter is an indwelling urinary catheter configured to be retained in the bladder of a patient.

### BRIEF DESCRIPTION OF THE FIGURES

The present invention will now be described by way of example with reference to the accompanying drawings. In the drawings:

FIG. 1 is an isometric view of an example of a shaft of a urinary catheter;

FIG. 2 is a cross-section of the shaft of the catheter of FIG. 1 on line A-A;

FIG. 3(a) is an isometric view of an example of a port unit of a urinary catheter;

FIG. 3(b) is another view of the port unit of FIG. 3(a);

FIG. 4 is another view of the port unit of FIG. 3(a) showing a proximal recess;

FIG. 5 is a cross-section of the distal part of a urinary catheter, with an uninflated balloon in place;

FIG. 6 is a cross-section of the distal part of the urinary catheter of FIG. 5, with a fully inflated balloon in place;

FIG. 7 shows exemplary steps of a method for manufacturing a urinary catheter;

FIG. 8 is a cross-section of the distal part of a urinary catheter where the internal wall of the balloon is bonded to the port unit with the balloon in an uninflated state;

FIG. 9 is a cross-section of the distal part of the urinary catheter of FIG. 8 with the balloon in an inflated state.

### DETAILED DESCRIPTION

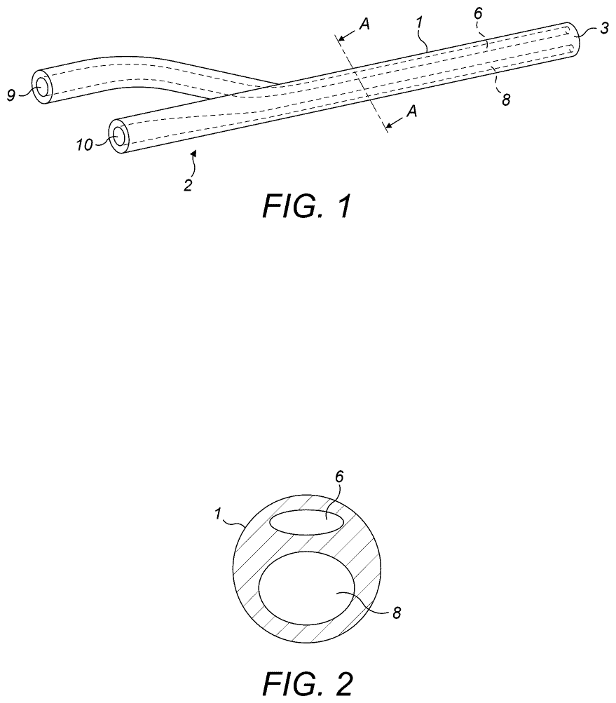

FIG. 1 shows an example of a shaft 1 for a urinary catheter. The catheter shaft 1 has a proximal end 2. The proximal end 2 is intended to sit outside the body when the catheter is in use. The shaft has a distal end 3. The distal end 3 is intended to sit inside the body of a user when the catheter is in use. The distal end 3 may sit in the bladder of a user when the catheter is in use. The shaft comprises an inflation lumen 6 which runs along the shaft. The shaft comprises a drainage lumen 8 which runs along the shaft. The distal end of the shaft may terminate at a plane perpendicular to the longitudinal axis of the shaft.

The shaft 1 of the catheter may be formed of a material such as polyurethane, a silicone elastomer or latex. The shaft 1 may be made from a thermoplastic material. The thermoplastic material may plastically deform when heated past a transition temperature. The shaft may be formed by extrusion.

In this example, the shaft comprises a proximal inflation opening 9 and a proximal drainage opening 10. A collecting vessel for urine can be attached to the drainage opening 10. Fluid can be introduced through the inflation opening 9 to then pass through the inflation lumen 6.

FIG. 2 shows a cross-section of the shaft on line A-A of FIG. 1, illustrating the lumens 6, 8. The inflation lumen 6 may have a smaller diameter than the drainage lumen 8. In FIG. 2, the lumens 6, 8 are shown as having ovular cross sections. The cross-sections of the lumens 6, 8 may have different shapes.

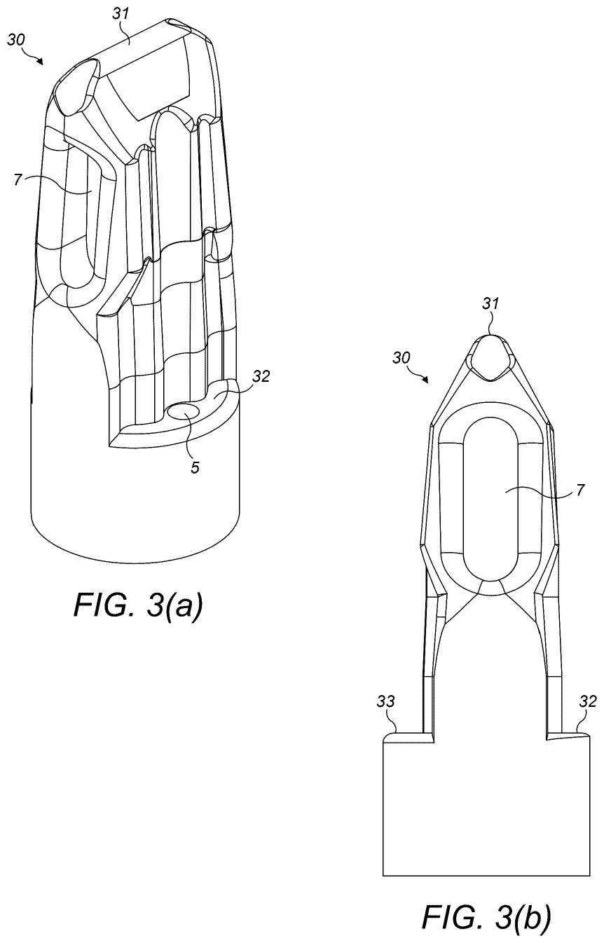

The catheter also comprises a port unit. FIG. 3(a), 3(b) and 4 show an example of a port unit 30. The port unit 30 is formed separately to the shaft 1. In other words, the port unit is not integrally formed with the shaft. The separate port unit is secured to the shaft. The port unit 30 may be a unitary component.

The port unit comprises one or more ports for communicating fluid between the body of the patient and the shaft (specifically a lumen of the shaft) when the catheter is in use.

The port unit may accommodate a balloon, as shown in the examples described herein. However, in other implementations, the catheter may not have a balloon, the port unit may not have an inflation port and the shaft may not have an inflation lumen.

The distal end 3 of the shaft 1 is received in a recess of the port unit 30 at the proximal end of the port unit, as will be described in more detail below. The outer diameter of the distal end of the shaft is less than the diameter of the proximal recess of the port unit. The distal end of the shaft may be secured in the recess of the port unit using a friction fit or, more preferably, using an adhesive.

The distal end of the port unit terminates in a tip 31. The tip may be tapered. This may allow for easier insertion to the urethra.

In this example, a drainage port 7 and an inflation port 5 are defined in the port unit of the catheter. The port unit may in some cases have more than one drainage port and more than one inflation port.

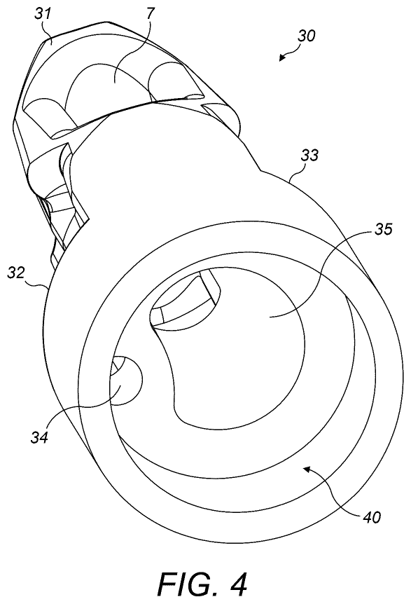

The inflation port 5 is intended for allowing the inflation of a balloon attached to the port unit. The inflation port 5 can communicate with an inflation lumen 6 which runs along the shaft. The inflation port is configured to communicate with the inflation lumen of the shaft via an inflation passage 34 in the port unit (see FIG. 4). The inflation port 5 may communicate directly with the inflation lumen 6 via passage 34, or as described below a separate connection tube may be inserted into the inflation port and the passage 34 that connects the interior of the balloon with the inflation lumen 6.

The drainage port 7 is intended for draining urine from the bladder of a user. The drainage port 7 communicates with the bladder of a user in use. The drainage port is configured to communicate with the drainage lumen of the shaft via a drainage passage 35 in the port unit. The drainage port 7 communicates with the drainage lumen 8 which runs along the shaft. There may be multiple drainage ports 7 in the distal end of the catheter. Preferably each drainage port 7 communicates with the drainage lumen 8. Preferably each drainage port 7 communicates with the drainage passage 35.

The or each inflation port 5 could be proximal of the drainage port 7, or of a subset of the drainage port 7 or of all the drainage ports 7. The or each inflation port 5 could be distal of the drainage port 7, or of a subset of the drainage ports 7 or of all the drainage ports 7.

As shown in FIG. 3(a), 3(b) and 4, in this example the port unit 30 comprises a first shoulder 32. The first shoulder 32 is recessed relative to the largest external diameter of the port unit at its proximal end. In other words, the radius of the port unit is reduced at the shoulder 32. In this example, the inflation port 5 is located on the first shoulder 32. The port unit 30 as comprises a second shoulder 33 on the opposite side of the port unit to the first shoulder 31. The second shoulder 33 is recessed relative to the largest external diameter of the port unit at its proximal end. In other words, the radius of the port unit is reduced at the shoulder 33. The balloon of the catheter may be secured to one or more of the first shoulder and the second shoulder. Because the shoulders are recessed, the balloon can conveniently be accommodated in this recess when uninflated, which may allow easier insertion of the catheter.

In other embodiments, the first shoulder and the second shoulder may be replaced with a continuous ledge around the entire circumference of the port unit. The ledge may be recessed relative to the largest external diameter of the port unit at its proximal end. In other words, the radius of the port unit is reduced at the ledge. This ledge may act as an adhesion point for one or more ends of the balloon 50. The inflation port 5 may be on the ledge.

As shown in FIG. 4, the port unit 30 comprises a proximal recess 40 for receiving the distal end of the catheter shaft. The proximal recess is at the proximal end of the port unit. In this example, the proximal recess 40 is in the form of a flange that receives the distal end of the shaft. The proximal flange hugs the distal end of the shaft. In this example, the recess has a circular cross section. The proximal recess has an internal diameter that is larger than the external diameter of the shaft. This may allow the shaft to be received in the proximal recess.

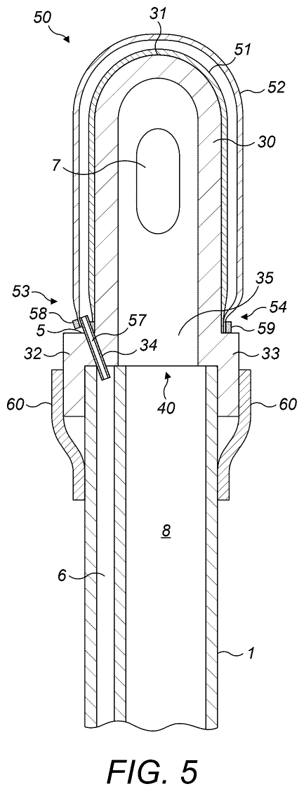

FIG. 5 is a cross-section of the catheter along the longitudinal axis of the shaft with an uninflated balloon, shown generally at 50, installed on the port unit 30.

The balloon 50 has an uninflated state. This may be the balloon's state when the catheter is packaged for supply to a user. The catheter having the balloon applied thereto in its uninflated state may be packaged in a sealed package whose interior is sterile. The balloon may initially adopt the uninflated state. In its uninflated state the exterior surface of the balloon may conform closely to the exterior surface of the tip. This may assist insertion of the catheter into a user.

The catheter of FIG. 5 has two drainage ports 7 in the port unit 30, one on each side of the port unit 30. In this example, the balloon 50 is generally in the form of a tube having an internal wall 51 and an external wall 52. The tube is generally elongate, extending between ends 53, 54. The balloon is made of an elastic sheet material. The tube constitutes a conduit part or all of whose walls are elastic and/or flexible. The balloon is sealed except for an aperture near one of its ends (end 53), by which the interior of the balloon can communicate with the inflation port 5, or as described below may be attached to an inflation connection tube 57.

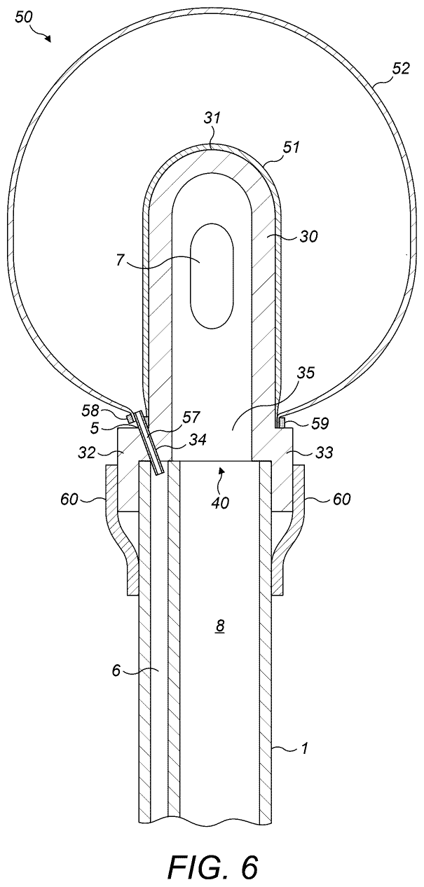

In its uninflated state the balloon extends over the distal tip 31 of the port unit 30. One or more regions of the balloon may be attached to the port unit 30 of the catheter. One region of attachment may surround the inflation port 5. The balloon may have an aperture in its wall facing the inflation port 5. The aperture may communicate with the inflation lumen 6. In this way the balloon can be sealed around the connection tube to permit pressure in the balloon to be increased by fluid flow through the inflation lumen of the shaft.

Where a connection tube is used for inflation of the balloon, as shown in FIGS. 5 and 6, the aperture of the balloon at end 53 of the balloon is sealed to one end of the connection tube 57. The balloon may be bonded to the tube 57 using an adhesive, shown at 58. The opposite end of the connection tube is placed inside the inflation lumen 6 of the shaft 1. As a result, the balloon can be inflated by introducing fluid such as water or air into the balloon through the inflation lumen 6. The tube-like form of the balloon extends over the tip 31 of the port unit. The balloon is bent around the tip. The end 54 of the balloon remote from the connection tube is also attached to the port unit. In this example, the end 54 of the balloon remote from the connection tube is attached to the port unit at the shoulder 33 using an adhesive 59. This holds the balloon 50 bent over the tip 31. The end 54 of the tubular balloon is sealed.

When the catheter is inserted into the patient, the urethra and bladder wall can push against and produce a friction force on the tip 31 of the port unit. As the balloon is located distally of the tip 31, this friction force may act on the balloon and result in the balloon being detached from the catheter. If the balloon is detached from the catheter, this can cause discomfort to the patient, and in the worst case result in the balloon being left in the bladder of the patient after removal of the catheter.

The balloon can be connected to the inflation port 5 by bonding the skin of the balloon directly to the inflation port. This may prevent the balloon from detaching from the port unit.

The example shown in FIGS. 5 and 6 uses a connection tube 57 which may further improve the reliability of the catheter. The connection tube conveniently directs fluid provided via the inflation lumen 6 of the shaft to the inside of the balloon 50, allowing it to inflate.

The connection tube 57 may be provided as a tube with an interior and exterior wall. The cross-section of the connection tube 57 may be circular, ovular, square, rectangular or any other shape. In this example, the connection tube 57 is generally circular, as this is the standard form of manufactured tube. The shape may vary depending on the design requirements for the catheter.

In the example shown in FIG. 5, the connection tube 57 is received in the inflation port 5 and passage 34 and protrudes from the inflation port 5. The connection tube 57 also protrudes into the interior of the balloon 50 through the aperture at one end of the balloon (e.g. end 53). The connection tube 57 protrusion can provide a mating surface for the interior of the balloon to be secured to. The balloon may be secured to the connection tube 57 using adhesive or any other suitable securing means. The mating surface of the connection tube 57 can provide additional area for the interior of the balloon to mate with the connection tube 57. This can provide a larger area for applying adhesive and consequently provide a more secure joint than if the balloon was directly attached to the inflation port 5. Preferably the connection tube 57 protrudes at least 2 mm from the inflation port 5 and protrudes at least 2 mm into the interior of the balloon to provide a sufficient mating surface. These dimensions may vary depending on the size of the catheter and the strength of the adhesive.

The connection tube 57 may also protrude through the inflation passage 34 and into the inflation lumen 6 of the shaft. This may provide a mating surface for the connection between the connection tube 57 and the inflation lumen 6. The connection tube 57 may be secured to the inflation lumen 6 using adhesive or any other suitable securing means. The mating surface of the connection tube 57 can provide additional area for the inflation lumen 6 to mate with the connection tube 57. This can provide a larger area for applying adhesive and consequently provide a more secure joint than if the balloon was directly attached to the inflation port 5. Preferably the connection tube 57 protrudes at least 2 mm into the inflation lumen 6 to provide a sufficient mating surface. These dimensions may vary depending on the size of the catheter and the strength of the adhesive.

In the example shown in FIGS. 5 and 6, the balloon is attached to the connection tube 57. In particular, the interior of the balloon is attached to the connection tube 57. The attachment may be provided by a friction fit or the attachment may be provided by bonding the connection tube 57 to the interior of the balloon, for example using an adhesive. It is preferable to use an adhesive, as this may provide a more secure attachment.

It can be seen in FIG. 5 that the largest external diameter of the port unit 30 at its proximal end is larger than the external diameter of the shaft to allow the recess of the port unit to receive the distal end of the shaft.

When the port unit 30 is secured to the distal end 3 of the shaft, the drainage lumen 8 is aligned with the drainage passage 35. This allows urine to drain from the bladder through drainage port 7, through drainage passageway 35 and into the drainage lumen 8. In implementations having a balloon, the inflation passage 34 may be aligned with the inflation lumen 6. The passage(s) may be fully or partially (i.e. at least some overlap) aligned with the lumen(s). The alignment may be parallel to the longitudinal axis of the catheter shaft 1. They may be aligned to allow fluid communication between the respective passage and the respective lumen. For example, to allow air to flow between the inflation lumen 6 and the balloon 50 (either via inflation passage 34 and out of inflation port 5 directly, or via a connection tube 57 installed in the inflation passage 34) and/or to allow the flow of urine between passage 35 and drainage lumen 8.

In the example shown in FIGS. 5 and 6, a heat shrink component 60 is used to smooth the transition region between the distal end of the shaft and the proximal end of the port unit. This may be in the form of a film or tube that is applied to the transition region. Heat may then be applied to shrink the film or tube to bond it to the distal end of the shaft. This may result in a tapered transition between the distal end of the shaft and the proximal end of the port unit of the catheter. This may improve comfort for the patient.

FIG. 6 is a cross-section of the catheter along the longitudinal axis of the shaft showing the fully inflated balloon. It should be noted that in its fully inflated state the balloon might be capable of further inflation (i.e. over-inflation). The fully inflated state is the state in which it would normally be left indwelling in a patient's bladder. In its fully inflated state, the size of the balloon, whose outer wall extends radially outward from the shaft of the catheter, resists withdrawal of the catheter through the urethra. This retains the distal end of the catheter in the bladder. The balloon can also form a seal at the base of the bladder to resist leakage of urine past the catheter.

In the example shown in FIGS. 5 and 6, the recess 40 of the port unit 30 surrounds the distal end of the shaft around an arc of 360 degrees about the longitudinal axis of the shaft. In other implementations, the recess of the port unit may surround the distal end of the shaft around an arc of more than 90, 180 or 240 degrees about the longitudinal axis of the shaft. The recess may hug the distal end of the shaft continuously or non-continuously (for example, with a castellated flange) about the longitudinal axis of the shaft.

Before the catheter is used, a reservoir containing a predetermined volume of fluid can be engaged with the inflation lumen 6 of the shaft. The reservoir could be a syringe or a bag. Once the tip of the catheter is in place in the bladder, the fluid can be squeezed from the reservoir into the balloon. The predetermined volume of fluid can be such as to cause the balloon to be fully inflated when the reservoir is fully evacuated. A valve may be provided in the inflation lumen 6 to resist fluid flow in the inflation lumen 6 towards the proximal end 2 of the shaft. This can help the balloon to remain inflated.

As noted above, the balloon is preferably in the form of an elongate tube folded over the tip 31 of the port unit 30. In this example, the ends 53, 54 of the tube are attached to the port unit on either side of the tip. In this example, the attachment points are proximal of, and on either side of, the drainage port 7. In other embodiments the attachment points may overlap the drainage port(s) 7 or be distal to them. When the balloon is inflated, the balloon material stretches.

The balloon may be attached to the port unit by adhesive, by welding (e.g. thermal welding) or by a mechanical fixing such as a collar configured to clamp one or more parts of the balloon to the exterior of the port unit.

In any of the embodiments, the outer surface of the port unit may define one or more recesses in which the uninflated balloon can sit, such as the shoulders 32, 33. The recess(es) may be sized so that the exterior of the uninflated balloon lies flush with the exposed surface of the port unit. This may help the catheter to be inserted through the urethra.

In the examples described herein, the first shoulder 32 and the second shoulder 33 provide a surface for the first region 53 and second region 54 of the balloon to be respectively attached to the port unit. As the first shoulder 32 and the second shoulder 33 are recessed relative to the largest external diameter of the port unit, the surface of the shoulders 32, 33 is provided normal to the longitudinal axis of the shaft 1. The first region 53 and the second region 54 of the balloon are provided at the ends of a tube. In this way, the first shoulder 32 and the second shoulder 3 provide a surface for the balloon first region 53 and second region 54 to be respectively attached to the port unit. By providing an attachment surface for at least one end of the tube that forms the balloon on the shoulders 32, 33 this can provide a larger and/or flatter mating surface for the connection. This may further increase the strength of the connection between the balloon and the port unit.

In FIG. 3(a), it is shown that the inflation port 5 is located on the first shoulder 32 of the port unit. The result of this is that the connection tube 57 can protrude from the first shoulder 32 and into the interior of the balloon. The combination of attaching the first region 53 of the balloon to the connection tube 57 attaching to the interior of the balloon is that the connection between the balloon and the inflation lumen 6 may be significantly strengthened. In some implementations, the tube 57 may also be secured to the inflation passage 34.

As mentioned above, the first shoulder 32 and the second shoulder 33 also provide a recess in the port unit for the first region 53 and the second region 54 of the balloon to be located in. As shown in FIGS. 3 and 4, the shoulders 32, 33 are recessed from the largest external diameter of the port unit (which in this example is at its proximal end) so that the first region 53 and the second region 54 of the uninflated balloon may also be located in line with (or flush with) or inside the largest external diameter of port unit. Providing the uninflated balloon inside the largest external diameter of the port unit may reduce the likelihood of protruding features catching on the urethra or the bladder wall when the catheter is inserted into the patient.

In this example, the greatest external diameter of the port unit is located at the proximal end of the port unit and the external diameter of the port unit tapers after this point. It is also possible for the port unit to be of constant diameter about the longitudinal axis of the catheter. It is preferable for the port unit to taper. In particular, it preferable for the taper to increase at the tip 31 to produce a rounded shape. The rounded shape has a number of advantages. Firstly, as mentioned above, the balloon may bend over the tip and a rounded shape may provide a continuous surface, without sharp edges, for the balloon to rest against. This way the balloon is less likely to be punctured by accident. Secondly, when the catheter is inserted into the patient, a rounded shape is less likely to catch the urethra and bladder walls. This may provide better comfort to the patient.

In this example, because the external diameter of the port unit at its tip is wider in one direction than in the direction normal to this, this can provide more frictional resistance to the balloon folded over the distal tip of the port unit, as there is a greater surface area of the distal tip in contact with the balloon along this direction.

There may be one, two or more drainage ports 7. Preferably, there is a drainage port 7 between each leg of the balloon as it extends along the side of the port unit. There may be one, two or more inflation ports 5. The balloon may be inflated from a single end or from more than one end.

In the examples shows in FIG. 3(a), 3(b) and 4-6, the drainage port 7 is located on a side of the port unit. The balloon extends from the first and second regions 53, 54 and over the tip 31 of the port unit. In this example the first and second regions 53, 54 are proximal of, and on either side, of the drainage port 7. In other embodiments the attachment points may overlap the drainage port(s) or be distal to them. The result of this is that the drainage port 7 is recessed relative to the inflated balloon. This may reduce the likelihood of the drainage process drawing the patient's bladder wall into the drainage port 7, which may cause significant discomfort and possible injury.

The port unit 30 may be injection moulded. Injection moulding may advantageously allow identical port unit components to be produced in large volumes. The injection moulded port unit can be secured to the shaft of the catheter and the balloon after formation. The port unit may be made of a silicone elastomer material. Other materials may alternatively be used.

Injection moulding may use a ram or screw-type plunger to force molten material into a mould cavity. The molten material solidifies into the shape of the contour of the mould. Moulds may have a single cavity or multiple cavities, allowing to form multiple different geometries in a single cycle. Moulds are generally made from steel, such as tool steel or stainless steel.

Raw material, for example in the form of pellets, may be fed through a hopper into a heated barrel with a reciprocating screw. Upon entrance to the barrel, the temperature increases, reducing the viscosity of the raw material and allowing it to flow. The screw delivers the raw material towards the mould. The screw may mix and homogenise the material and/or reduce the required heating time by mechanically shearing the material and/or adding a significant amount of frictional heating. The material feeds forwards through a valve and collects at the front of the screw into a volume, which is generally referred to as a shot. A shot is the volume of material that is used to fill the mould cavity, including compensating for shrinkage, and providing a cushion to transfer pressure from the screw to the mould cavity. When enough material has gathered, the material is forced at high pressure and velocity into the part-forming cavity of the mould.

Once the screw reaches the transfer position, a packing pressure is applied, which completes mould filling and compensates for thermal shrinkage. The packing pressure is applied until the gate (cavity entrance) of the mould solidifies. Once the gate solidifies, no more material can enter the cavity.

The screw then reciprocates and acquires material for the next cycle, while the material within the mould solidifies and cools. Once the required temperature has been achieved, the mould is opened. The moulded port unit can then be removed. The mould can then be re-closed and the process can be repeated.

Where the port unit is made from a silicone elastomer, the port unit may be produced using liquid injection moulding. Liquid silicone can be introduced under pressure into the mould cavity and then heat and/or moisture cured.

Once the port unit has been formed, the catheter can be assembled by securing the port unit to the distal end of a catheter shaft.

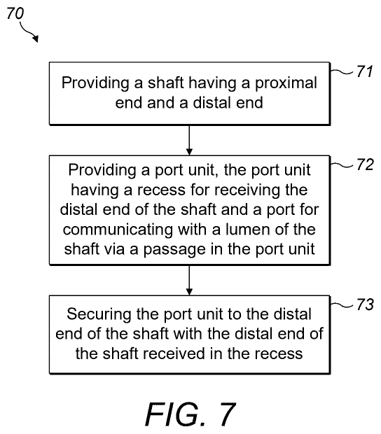

FIG. 7 shows the exemplary steps of a method for manufacturing a catheter. At step 71 the method comprises providing a shaft having a proximal end and a distal end. At step 72, the method comprises providing a port unit, the port unit having a recess for receiving the distal end of the shaft and a port for communicating with a lumen of the shaft via a passage in the port unit. At step 73, the method comprises securing the port unit to the distal end of the shaft with the distal end of the shaft received in the recess.

The port unit can be presented to the distal end of the shaft with an adhesive on the surface of the recess to bond the distal end of the shaft and the port unit together.

In embodiments where the catheter comprises a connection tube 57, the connection tube with the balloon 50 secured to it at one end of the tube 57 may be attached to the inflation passage 34 of the port unit before bonding the port unit to the distal end of the shaft. The connection tube protrudes from the inflation port. The use of the connection tube can allow the bonding between the shaft and the port unit without the inflation route becoming blocked, for example by adhesive.

The method shown in FIG. 7 may further comprise one or more of the following steps: providing a balloon in the form of an elongate tube; providing a connection tube; sealing the elongate tube at a first end; sealing the elongate tube around the connection tube at a second end; advancing the connection tube through a hollow channel of the port unit to connect the connection tube with an inflation lumen of the shaft; and bonding the connection tube to the inflation lumen.

The method may further comprise applying a heat shrink component to the transition between the distal end of the shaft and the port unit. The heat shrink layer may be positioned over the outer surfaces of the port unit and the distal end of the shaft at the transition region. The method may further comprise applying heat to the heat shrink component to shrink it over the transition between the shaft and the port unit so as to smooth the transition. This may avoid having a stepped transition between the distal end of the shaft and the port unit, which may improve comfort during insertion.

The transition region may in some examples extend on either side of the longitudinal position where the shaft meets the port unit. The heat shrink component 60 may be applied to this region, as shown in FIGS. 5 and 6. In other examples, the heat shrink component may be applied proximally of the longitudinal position where the shaft meets the port unit. In this example, the transition region is proximal of the longitudinal position where the shaft meets the port unit.

There may be a bonding layer between the shaft and the port unit. The bonding layer may comprise an adhesive. The bonding layer may comprise a zone of melted or softened material by which the shaft and the port unit have been heat welded together.

Where the balloon of the catheter is in the form of an elongate tube, the balloon may be prepared for attachment to the port unit as follows. The method for manufacturing the urinary catheter may comprise one or more of these steps. The elongate tube of the balloon may be cut from a length of tube. The length of the balloon tube may be, for example 25-30 mm. Adhesive may be dispensed into a first end of the tube. The first end of the tube may be clamped to close the first end of the tube. A rod may be inserted into a second end of the tube. The rod may have a diameter of, for example, 0.5-1 mm, for example 0.9 mm. The rod may be centred in the second end of the tube. Adhesive may be dispensed into the second end of the tube around the rod. The adhesive can be cured. For example, where the adhesive is a silicone adhesive, the adhesive may be moisture cured. The rod can be removed from the adhesive at the second end of the tube once cured. This can form a channel for a connection tube to be inserted into the second end of the tube.

In some implementations, an adhesive may be applied to one or more of (i) the recess of the port unit and (ii) the distal end of the shaft. The distal end of the shaft can then be inserted into the recess of the port unit. This may cause a layer of adhesive to surround the shaft at the base of the port unit when the shaft is inserted into the recess.

The method for manufacturing the urinary catheter may comprise applying an adhesive to one or more of the distal end of the shaft and recess of the port unit. The method may comprise securing the port unit to the distal end of the shaft with the distal end of the shaft received in the recess.

When using a connection tube, the connection tube may be installed in the inflation lumen of the shaft or in the inflation passage of the port unit before the adhesive is applied to secure the port unit to the distal end of the shaft. The connection tube may have a length of approximately 6-10 mm. Where the connection tube is inserted into the inflation lumen of the shaft, it may be inserted to a depth of approximately 4-8 mm into the inflation lumen from the distal end of the shaft. When the port unit is secured to the shaft, the connection tube may be guided through the inflation passage of the port unit to align the shaft and the port unit.

The connection tube may be in an interference fit with the inflation lumen of the shaft. The external diameter of the connection tube may be larger than the diameter of the inflation lumen. The interference fit may be sufficiently tight such that when the catheter is in use, water used to inflate the balloon does not leak out between the connection tube and the diameter of the inflation lumen. This interference fit can allow the connection tube to move within the inflation lumen if, for example, a force is applied to the proximal end of the catheter shaft, causing the catheter to stretch while the balloon is inflated to anchor it in the bladder.

To smooth the transition between the shaft and the base of the port unit, heat shrink tubing may be applied over a junction region where the shaft meets the port unit, over the layer of adhesive. The heat shrink tubing can be heated (for example using a heat gun) to shrink the tubing around the junction region and the layer of adhesive. This can spread the layer of adhesive evenly around the junction region. The adhesive can then be cured (for example, moisture cured). The heat shrink tubing can be removed. This can smooth the transition between the shaft and the port unit. This may allow for improved insertion of the catheter.

The method for manufacturing the urinary catheter may comprise, subsequent to securing the port unit to the distal end of the shaft using adhesive, one or more of the following steps: applying a heat shrink tubing over the junction region where the distal end of the shaft meets the port unit; heating the heat shrink tubing to shrink the heat shrink tubing so as to smooth a layer of the adhesive at the junction region; curing the adhesive; and removing the heat shrink tubing.

Heat shrink tubing can also be used during the attachment of the ends of the tube of the balloon to the port unit. For example, heat shrink tubing can be used to hold the ends of the balloon in place while adhesive is applied and/or cured.

In one example, the second end of the tube of the balloon may be slid into place over the connection tube. The connection tube may protrude from the first shoulder 32 of the port unit. Heat shrink tubing may be slid over the port unit, for example such that the top of the heat shrink tube is at the base of the drainage lumen and/or the bottom of the heat shrink tube is at the base of the port unit. Adhesive may be applied to one or more shoulders 32, 33 of the port unit. The elongate tube of the balloon can be bent over the tip of the port unit and the first end of the tube secured to the second shoulder 33 of the port unit. The heat shrink tubing can be heated to shrink the tubing around the port unit and the ends of the tube of the balloon. The adhesive can then be cured (for example, moisture cured). The heat shrink tubing can then be removed. This can ensure that the ends of the balloon tube are securely bonded to the port unit. The method for manufacturing the urinary catheter may comprise: applying a heat shrink tubing over the port unit; applying adhesive to one or more of the first and second ends of the elongate tube of the balloon to bond the one or more ends to the port unit; heating the heat shrink tubing to shrink the heat shrink tubing; curing the adhesive; and removing the heat shrink tubing.

In some implementations, where the balloon is bonded to the port unit, the balloon tube may be bonded to the port unit between the first and second ends. The balloon tube may be bonded to the port unit using, for example, a silicone adhesive. The balloon tube may be bonded to the port unit along its entire length between the first and second ends (or, for example, at least 50, 60, 70, 80, or 90% of the length of the balloon). In some implementations, where the balloon is bonded to the port unit, the entire length of the internal wall 51 of the balloon tube may be bonded to the port unit (or, for example, at least 50, 60, 70, 80, or 90% of the length of the internal wall 51 of the balloon).

When the balloon is inflated, stresses in the tube of the balloon can cause stress to be applied to the port unit. This can cause the port unit to deform and pull open the drainage ports 7. The bonding of the balloon to the port unit in this way can result in the drainage ports being pulled open wider when the balloon is inflated, giving the catheter an improved ability to drain urine, as well as sediment and other debris from the bladder.

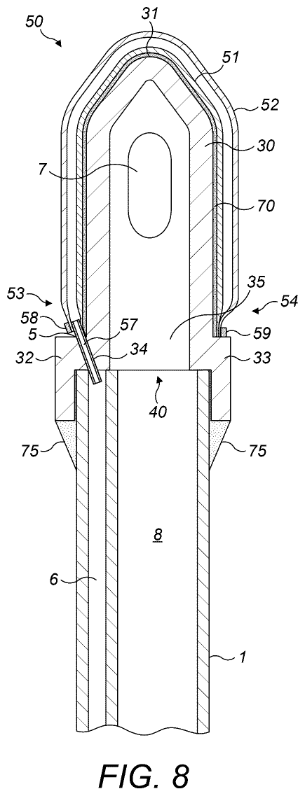

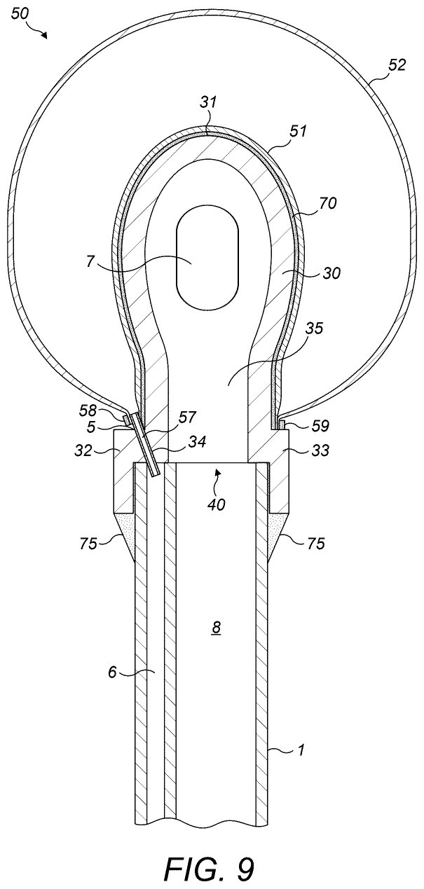

FIGS. 8 and 9 show such a catheter with the balloon in uninflated and inflated forms respectively. FIG. 8 shows the balloon 50 in a generally uninflated state bonded to the port unit along the length of its inner wall 51. FIG. 9 shows the balloon 50 in an inflated state. The layer of adhesive between the port unit 30 and the length of the internal wall 51 of the balloon 51 is shown at 70. FIGS. 8 and 9 also show a smoothed layer of adhesive 75 at the junction region where the distal end of the shaft meets the port unit, as described above.

The material of the port unit may have suitable properties that allow for the deformation of material around the drainage ports. For example, the material of the port unit may have a Shore Hardness of approximately 30-40 durometer. This can allow the material of the port unit to deform to allow the drainage ports to widen, whilst still allowing sufficient rigidity of the port unit for insertion into a patient.

The catheter manufacture approach described herein allows an injection moulded port unit to be attached to an extruded shaft. The port unit and the shaft may both be made of a material comprising a silicone elastomer. Injection moulded port units can allow for high repeatability with low manufacturing tolerances and a smooth surface finish. A rough surface finish can result in discomfort to the patient upon insertion and/or may increase the likelihood of bacteria adhering to the catheter. Smooth surface finish may offer improved comfort to the patient during insertion of the catheter. This approach may allow the major components of the catheter such as the shaft and the tip to be fabricated separately (for example, one or more parts being made from a silicone elastomer) and secured together (for example using a silicone adhesive).

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description, it will be evident to a person skilled in the art that various modifications may be made within the scope of the invention.

What is claimed is:

1. A urinary catheter comprising:
a shaft having a proximal end and a distal end, the shaft having a lumen extending between the proximal end and the distal end; and
a port unit secured to the distal end of the shaft, the port unit comprising a port configured to communicate with the lumen of the shaft via a passage in the port unit, the distal end of the shaft being received in a recess of the port unit.

2. The urinary catheter as claimed in claim 1, wherein the catheter further comprises a balloon installed on the port unit.

3. The urinary catheter as claimed in claim 2, wherein the balloon is in the form of an elongate tube.

4. The urinary catheter as claimed in claim 2, wherein at least part of the balloon is located proximally of the port.

5. The urinary catheter as claimed in claim 1, wherein the distal end of the shaft terminates at a plane perpendicular to the longitudinal axis of the shaft.

6. The urinary catheter as claimed in claim 1, wherein the port is a drainage port and the lumen is a drainage lumen of the shaft.

7. The urinary catheter as claimed in claim 1, wherein the port is on a side of the port unit.

8. The urinary catheter as claimed in claim 1, wherein the port unit comprises an inflation port configured to communicate with an inflation lumen of the shaft via an inflation passage in the port unit.

9. The urinary catheter as claimed in claim 1, further comprising an adhesive for securing the shaft to the port unit.

10. The urinary catheter as claimed in claim 1, wherein the port unit is formed of a material comprising a silicone elastomer.

11. The urinary catheter as claimed in claim 1, wherein the port unit is an injection moulded component.

12. The urinary catheter as claimed in claim 1, wherein the recess of the port unit comprises a proximal flange hugging the distal end of the shaft.

13. The urinary catheter as claimed in claim 1, wherein the recess of the port unit surrounds the distal end of the shaft around an arc of more than 180 degrees about the longitudinal axis of the shaft.

14. The urinary catheter as claimed in claim 1, wherein the distal end of the shaft extends into the recess of the port unit by at least 2 mm.

15. The urinary catheter as claimed in claim 2, wherein the balloon is in the form of an elongate tube and is secured to the port unit between first and second ends of the elongate tube and/or wherein the balloon is secured to the port unit along at least 50% of the length of an internal wall of the balloon.

16. A port unit for a urinary catheter, the port unit comprising:
a recess configured to receive a catheter shaft; and
a port configured to communicate with a lumen of the catheter shaft via a passage in the port unit.

17. A method for manufacturing a urinary catheter comprising:
providing a shaft having a proximal end and a distal end;
providing a port unit, the port unit having a recess for receiving the distal end of the shaft and a port for communicating with a lumen of the shaft via a passage in the port unit; and
securing the port unit to the distal end of the shaft with the distal end of the shaft received in the recess.

18. The method as claimed in claim 17, wherein the method further comprises:
providing a balloon in the form of an elongate tube;
providing a connection tube;
sealing the elongate tube at a first end;
sealing the elongate tube around the connection tube at a second end;
advancing the connection tube through an inflation passage of the port unit to connect the connection tube with an inflation lumen of the shaft; and
bonding the connection tube to the inflation lumen.

19. The method of claim 17, wherein the method further comprises shrinking a heat shrink component over a transition between the shaft and the port unit so as to smooth the transition.

20. The method of claim 17, wherein the method further comprises, prior to securing the port unit to the distal end of the shaft, forming the shaft by extrusion and/or forming the port unit by injection moulding.
